(19) United States
(12) Patent Application Publication
(10) Pub. No.: US 2005/0246008 A1
(43) Pub. Date: Nov. 3, 2005

(54) Delivery system for vascular prostheses and methods of use

(75) Inventors: Michael Hogendijk, Santa Clara, CA (US); Eric Leopold, Redwood City, CA (US); Tim Huynh, Santa Clara, CA (US); Miles Alexander, Fremont, CA (US)

Correspondence Address: LUCE, FORWARD, HAMILTON & SCRIPPS LLP, 11988 EL CAMINO REAL, SUITE 200, SAN DIEGO, CA, 92130, US

(73) Assignee: NovoStent Corporation, Santa Clara, CA (US)

(21) Appl. No.: 10/925,756

(22) Filed: Aug. 25, 2004

Related U.S. Application Data
(63) Continuation-in-part of application No. 10/836,909, filed on Apr. 30, 2004.

(51) Int. Cl. A61F 2/06

(52) U.S. Cl. 623/1.11

(57) ABSTRACT

The present invention is directed to a delivery system for delivering a vascular prosthesis within a vessel, the vascular prosthesis having a contracted delivery configuration and a deployed configuration. The delivery system comprises a loader tube having a lumen preloaded with a delivery wire carrying a vascular prosthesis in the contracted delivery configuration. A separately inserted sheath includes a lumen configured to accept the vascular prosthesis, while retaining it in the contracted delivery configuration. The delivery wire is used to translate the vascular prosthesis to a distal end of the sheath for deployment in a vessel.

### REFERENCE TO RELATED APPLICATIONS

The present application is a continuation-in-part application of co-pending, commonly assigned U.S. patent application Ser. No. 10/836,909, filed Apr. 30, 2004.

### FIELD OF THE INVENTION

The present invention relates to a two-part delivery system for implantable vascular prostheses, wherein the delivery system provides reduced profile and enhanced flexibility to negotiate narrow vessels and tortuous anatomy.

### BACKGROUND OF THE INVENTION

Vascular stenting has become a practical method of reestablishing blood flow to diseased vasculature. Conventional stent delivery systems have problems negotiating vessels having reduced diameters and vessels that require tortuous or challenging anatomy to be traversed. Today there are a wide range of intravascular prostheses on the market for use in the treatment of aneurysms, stenosis, and other vascular irregularities. Balloon expandable and self-expanding stents are well known for restoring patency in a stenosed vessel, e.g., after an angioplasty procedure, and the use of coils and stents are known techniques for treating aneurysms.

Previously-known vascular prostheses and stents generally are retained in a contracted delivery configuration on or within a delivery system, which typically includes a guide wire, delivery catheter and sheath. Alternatively, the delivery system may include a catheter that includes one or more locking mechanisms that retain the stent on the catheter until it is desired to deploy the stent.

U.S. Pat. No. 4,665,918 to Garza provides a typical example of a delivery system for a self-expanding stent, and includes an inner member and sheath that cooperate to define a compartment that holds the stent in a contracted delivery configuration. The inner member includes a guide wire lumen that permits the delivery system to be advanced along a pre-positioned guide wire. Once positioned at the desired location within a vessel, the inner member is held stationary, while the sheath is retracted proximally, thereby permitting the stent to self-expand.

U.S. Pat. No. 4,733,665 to Palmaz describes a typical previously-known delivery system for a balloon expandable stent, that includes a balloon catheter and sheath. The stent is compressed onto the balloon of the balloon catheter; the sheath ensures that the stent does not come free from the catheter until the stent is located at the desired location within the vessel.

Due to the increased profile associated with employing a sheath to retain the stent on the delivery system, many previously-known delivery systems sought to eliminate the sheath. For example, U.S. Pat. No. 5,314,444 to Gianturco describes a delivery system wherein the stent is tightly compressed onto the balloon of the balloon catheter, whereby the sheath was omitted. Similarly, U.S. Pat. No. 4,553,545 to Maass and U.S. Pat. No. 5,147,370 to McNamara describe delivery systems for self-expanding helical stents that employed locking members disposed within the catheter to lock the ends of the stent in place until the stent was maneuvered through the vessel to its destination.

While such previously-known systems eliminated the sheath of the delivery system, the use of locking mechanisms required that the diameter of the catheter increase, so that little overall reduction in delivery profile was accomplished. Likewise for balloon expandable stent delivery systems, the ability to reduce the overall profile of the delivery system was limited by of thickness of the stent compressed onto the deflated balloon, the balloon inflation lumen diameter and guide wire lumen diameter, and need to make the inflation lumen walls sufficiently thick to withstand the inflation pressures required to deploy the stent.

For the foregoing reasons, even the best previously-known stent delivery systems generally have been limited to a minimum diameter of about 6 French. In addition, as noted above, previously-known delivery systems employ a layering of the sheath (if present), stent and inner member or balloon catheter. Notwithstanding the development of improved materials over the last two decades, the overall rigidity of the combined stent and delivery system has remained relatively high. This in turn has limited the ability to access smaller vessels and negotiate highly tortuous anatomy.

In addition to the foregoing drawbacks of previously-known stent delivery systems, the acceptance of self-expanding stents has been limited by problems peculiar to the design of such stents. Specifically, self-expanding stents may experience large length changes during expansion (referred to as “foreshortening”) and may shift within the vessel prior to engaging the vessel wall, resulting in improper placement.

Where the stent has a helical coil configuration, as described for example in PCT Publication WO 00/62711 to Rivelli, friction between the turns of the stent and the sheath or between individual turns of the stent, may cause the turns to bunch up, or overlap with one another, during deployment. U.S. Pat. No. 4,768,507 to Fischell et al. and U.S. Pat. No. 6,576,006 to Limon et al., each describe the use of a groove disposed on an inner member of the delivery system to prevent such axial movement, but such arrangements detrimentally increase the profile of the delivery system. Moreover, those delivery systems do not address the issue of stent foreshortening.

In view of the aforementioned drawbacks of previously-known stent delivery systems, it would be desirable to provide a delivery system and methods that provide a reduced profile, thereby enabling the delivery system to negotiate small diameter vessels.

It also would be desirable to provide a delivery system and methods that provide low rigidity in the delivery configuration, thereby allowing the delivery system to negotiate highly tortuous anatomy.

It further would be desirable to provide a stent delivery system for self-expanding stents and methods of use that provide a desired degree of foreshortening (including zero foreshortening) of the stent during deployment.

### SUMMARY OF THE INVENTION

In view of the foregoing, it is an object of the present invention to provide a delivery system and methods that provide a reduced profile, thereby enabling the delivery system to negotiate small diameter vessels.

It is another object of this invention to provide a delivery system and methods that provide low rigidity in the delivery configuration, thereby allowing the delivery system to negotiate highly tortuous anatomy.

It is a further object of the present invention to provide a stent delivery system for self-expanding stents and methods of use that provide a desired degree of foreshortening (including zero foreshortening) of the stent during deployment.

In accordance with the principles of the present invention, a two-part delivery system is provided that includes a loader tube/delivery wire component (preloaded with a stent) and a separately inserted sheath. In a preferred embodiment, the stent or other implantable device is compressed onto the delivery wire and retained in a contracted delivery configuration by the loader tube. The delivery wire preferably has a diameter in a range of 0.014 to 0.035″, and may be constructed in a manner similar to conventional guide wires. The loader tube preferably is relatively short, e.g., 10 cm, and is disposed adjacent to the distal end of the delivery wire.

In one preferred embodiment, the sheath is constructed of a thin-walled material with a non-stick interior liner, e.g., such as polytetrafluoroethylene, and has the same inner diameter as the inner diameter of the loader tube. This permits that loader tube to be coupled to the sheath so that the stent may be transferred from the loader tube to the sheath while the stent is retained in the contracted delivery configuration. Because the stent is not stored in the sheath, as in previously known systems, but only passes in a transitory manner through the sheath during delivery, the wall thickness of the sheath may be substantially thinner than in previously known delivery systems and substantially more flexible.

In accordance with a further aspect of the invention, the sheath is configured to be inserted to a desired position into a vessel along a conventional pre-placed guide wire. Once the sheath is positioned, the conventional guide wire is withdrawn. The delivery wire then is inserted into the proximal end of the sheath, and the loader tube is coupled to the proximal end of the sheath. The delivery wire (and attached stent) then are advanced from the loader tube through the sheath. Once the stent is located at a desired position within a vessel, the delivery wire is held stationary and the sheath is retracted to deploy the stent.

The foregoing method of the present invention thus permits the sheath to be separately advanced through highly tortuous anatomy. Because the sheath does not contain the stent when originally advanced through the patient's vessel, it is much less rigid than previously-known delivery systems. In addition, once the distal end of the sheath is inserted to a desired location within a vessel, the loader tube permits the stent to be pushed into and through the sheath in the contracted state. This feature ensures that there is no increase in the profile of the delivery system, and permits stents of the present invention to be delivered using sheaths as small as 3 French.

According to a further aspect of the invention, especially for use with helical ribbon stents, the delivery wire includes a winding section dimensioned to receive the stent. The winding section preferably comprises a guide that defines a pitch of the stent to facilitate consistent and accurate winding of the helical portion of the stent around the delivery wire. The winding section preferably is configured to provide zero or a desired degree of foreshortening, so that the length of the stent undergoes a predictable amount of change during deployment.

### BRIEF DESCRIPTION OF THE DRAWINGS

Further features of the invention, its nature and various advantages will be more apparent from the accompanying drawings and the following detailed description of the preferred embodiments, in which:

FIG. 1 is a view of an exemplary vascular prosthesis suitable for use with the delivery system of the present invention;

FIG. 2 is an exploded sectional view of a delivery system constructed in accordance with the principles of the present invention;

FIGS. 3A to 3E are side sectional views depicting use of the delivery system of FIG. 2 to treat a lesion in a patient's vessel;

FIG. 4 is a drawing depicting foreshortening of a ribbon-type stent as encountered with previously-known delivery systems as the stent expands from a contracted delivery configuration to an expanded deployed configuration;

FIG. 5 is a drawing depicting a ribbon-type stent unrolled to a flat configuration and projected onto an expanded deployed configuration (for clarity, only a single turn is shown, although it will be understood that in the deployed configuration the stent includes multiple turns); and

FIG. 6 is a drawing depicting trigonometric relationships between the wrap angle of the stent of FIG. 5 and width of the stent.

### DETAILED DESCRIPTION OF THE INVENTION

The present invention is directed to a delivery system for use with implantable vascular prostheses for a wide range of applications, such as treating aneurysms, maintaining patency in a vessel, and allowing for the controlled delivery of therapeutic agents to a vessel wall. In a preferred embodiment, the delivery system is configured for use with a stent having a helical ribbon portion joined, at its distal end, to a radially self-expanding anchor portion, such as depicted in FIG. 1.

Referring to FIG. 1, an exemplary stent for use with the delivery system of the present invention is described. As used in this specification, the terms “vascular prosthesis” and “stent” are used interchangeably. Vascular prosthesis 10 comprises helical section 12 and distal section 14, each capable of assuming contracted and deployed states. In FIG. 1, helical section 12 and distal section 14 are each depicted in the deployed state.

Vascular prosthesis 10 preferably is formed from a solid tubular member comprising a shape memory material, such as nickel-titanium alloy (commonly known in the art as Nitinol). The solid tubular member then is laser cut, using techniques that are per se known in the art, to a desired deployed configuration, as depicted in FIG. 1. An appropriate heat treatment, per se known in the art, then may be applied to vascular prosthesis 10 while the device is held in the desired deployed configuration (e.g., on a mandrel), thus conferring a desired deployed configuration to vascular prosthesis 10 when self-deployed.

In the illustrated embodiment, distal section 14 has a generally zig-zag configuration in the deployed state, wherein the zig-zag configuration preferably is formed by laser cutting a solid tube to form a pattern comprising plurality of arcuate struts 18 joined at apices 20. Distal section 14 is designed to be deployed from the delivery catheter of the present invention first to fix the distal end of the stent at a desired known location within a vessel. In this manner, subsequent deployment of helical section 12 of the stent may be accomplished with greater accuracy.

Helical section 12 preferably comprises a helical mesh configuration that includes a plurality of substantially flat turns 22. Plurality of turns 22 may include a multiplicity of openings, as illustrated by openings 24. It should be understood that the configuration of helical section 12 depicted in FIG. 1 is merely illustrative, and other patterns may be advantageously employed. Helical section 12 is coupled to distal section 14 at junction 26.

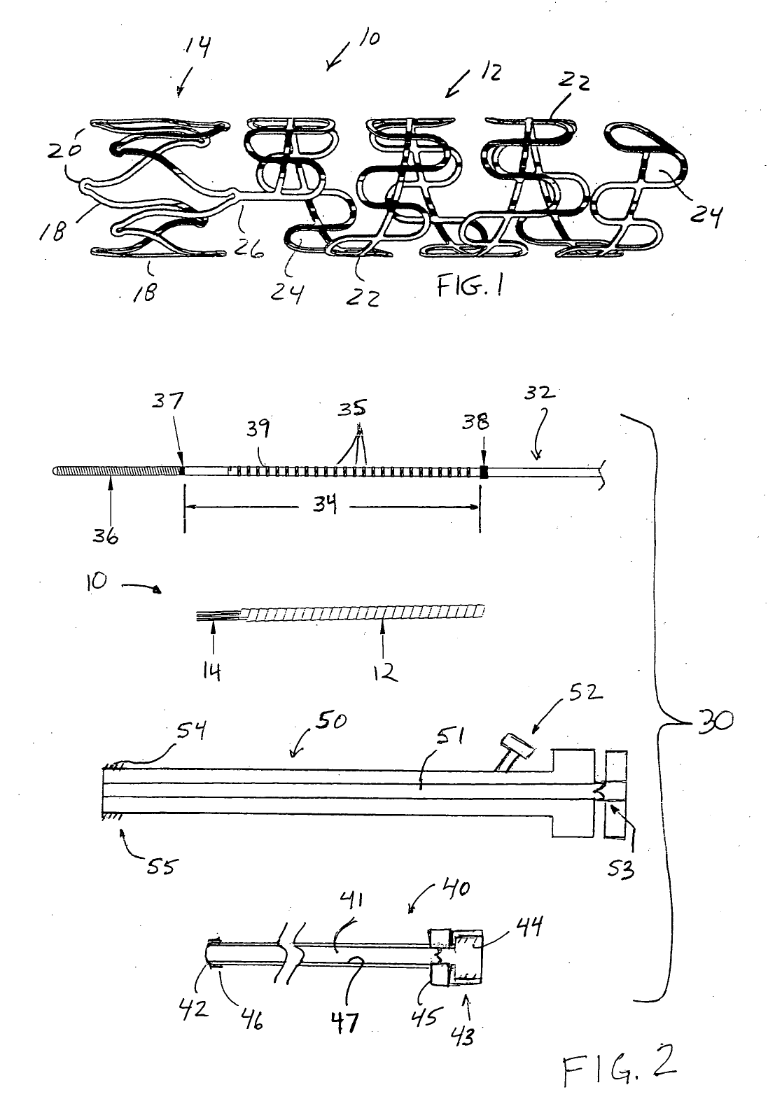

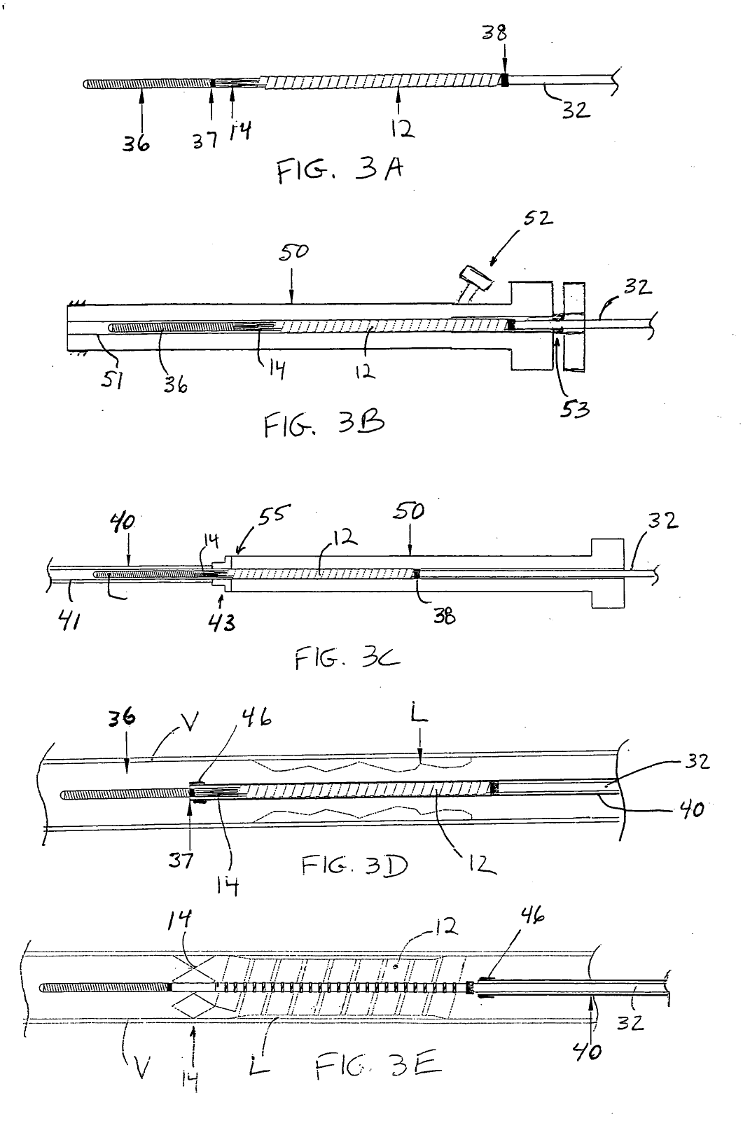

Referring to FIG. 2, a delivery system constructed in accordance with the principles of the present invention, suitable for use in delivering stent 10, is described. Delivery system 30 comprises delivery wire 32, sheath 40 and loader tube 50. Stent 10 is compressed onto delivery wire 32 as described hereinbelow.

Delivery wire 32 may comprise a conventional guide wire more than 100 cm in length (e.g., 120 cm) and having a diameter in a range of about 0.014 to 0.035″. In a preferred embodiment, the delivery wire further comprises winding section 34 at its distal end including guide 35. Guide 35 defines a pitch that facilitates consistent and accurate winding body portion 12 of the vascular prosthesis around delivery wire 32. Delivery wire 32 preferably includes atraumatic coil tip 36, distal marker 37 adjacent to coil tip 36 and proximal marker 38. Distal marker 37 is radiopaque and may be used to identify the location of the distal end of the stent under fluoroscopic guidance. Proximal stop 38 also preferably is radiopaque, and provides an abutment surface against which the proximal end of the stent may engage during retraction of sheath 40.

Winding section 34 corresponds to the length spanned by guide 35 between distal marker 37 and proximal stop 38. The winding section is dimensioned to receive vascular prosthesis 10, which in FIG. 2 is shown in a contracted delivery configuration. Guide 35 of winding section 34 defines helical ledge 39 that controls foreshortening of the stent during deployment, and may comprise a helical coil affixed to the outside diameter of the delivery wire, a larger diameter thread braided into a matrix of wires comprising the delivery wire, or may be formed by grinding a reduced-diameter helical groove into the exterior surface of the delivery wire.

Still referring to FIG. 2, sheath 40 comprises a thin-walled catheter having central lumen 41, atraumatic distal tip 42 having radiopaque marker 46, and proximal end 43 including luer-type coupling 44 and hemostatic valve 45. Sheath 40 preferably has a length of about 120 cm, and a diameter of 3 French, and includes non-stick interior liner 47 comprising, e.g., polytetrafluoroethylene. Hemostatic valve 45 may be of conventional construction, and permits delivery wire 32 and stent 10 to pass through it when opened, while substantially sealing the proximal end of the sheath when the valve is closed. As described in greater detail below, sheath 40 comprises a flexible material, such as used in catheters, e.g., polyethylene, polypropelene, etc., and may be inserted over a conventional pre-placed guide wire to negotiate tortuous anatomy.

Loader tube 50 comprises a substantially cylindrical tube having lumen 51, side port 52, optional hemostatic valve 53, and luer-type coupling 54 at distal end 55. Loader tube 50 comprises a relatively rigid material, such as polycarbonate and has a length of approximately 10 cm. In accordance with the principles of the present invention, lumen 51 has an inner diameter selected to retain vascular prosthesis 10 compressed about delivery wire 32. The inner diameter of lumen 51 is substantially equal to the inner diameter of lumen 41 of sheath 40. When is it desired to place vascular prosthesis 10, it may be pushed, still in the contracted state, from loader tube 50 and into and through sheath 40 using delivery wire 32. Non-stick liner 47 of sheath 40 facilitates movement of the stent between loader tube 50 and sheath 40.

Coupling 44 of sheath 40 accepts coupling 54 of loader tube 50 to enable transfer of the contracted stent from the loader tube into sheath 50. Illustratively, coupling 44 comprises a threaded section that mates with threads disposed on coupling 54 of loader tube 50. Alternatively, the couplings may comprise conventional luer-type connectors.

Hemostatic valves 45 and 53 prevent excessive backflow through the proximal ends of the sheath and loader tube, respectively, during coupling of the two components and advancement of the stent and delivery wire. Hemostatic valves 45 and 53 comprise conventional valve bodies having perforated elastomeric disks that self-seal under compression. Side port 52 of loader tube 50 permits an irrigation fluid, such as saline, or fluoroscopic dye to be introduced during stent delivery for diagnostic purposes.

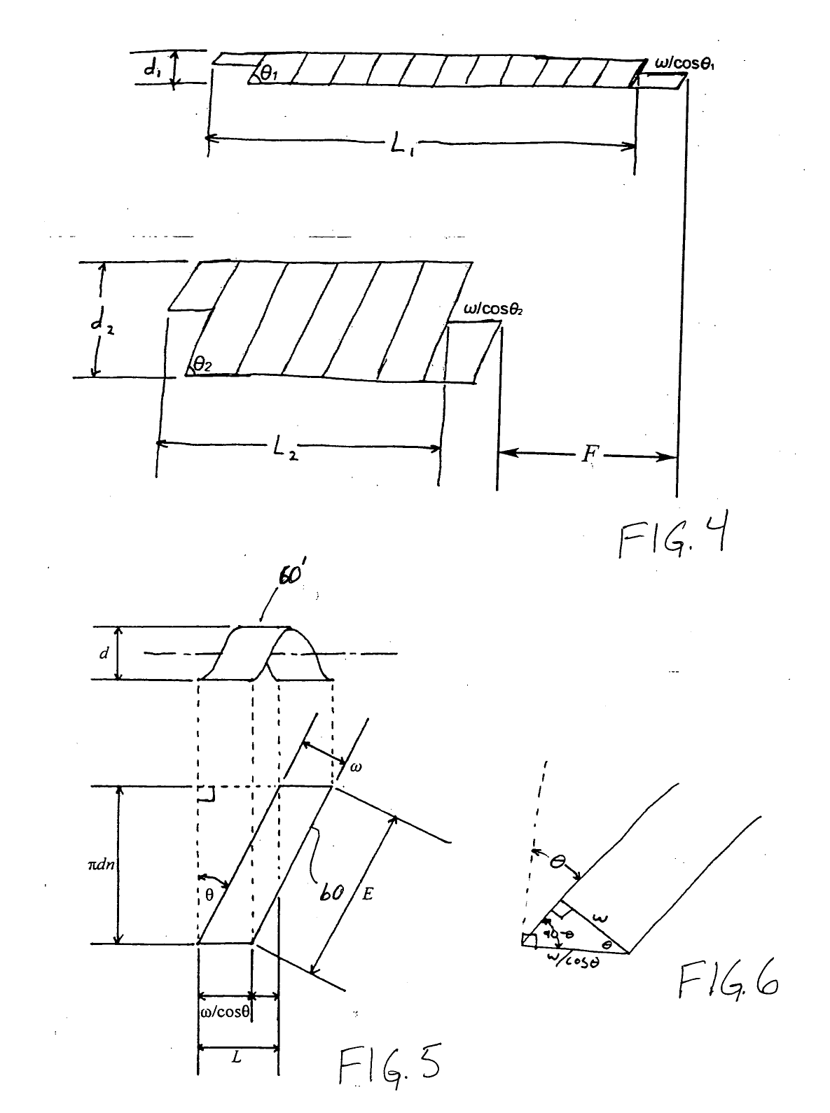

Referring now to FIGS. 3A-3E, a method of using the delivery system of FIG. 2 to deliver a vascular prosthesis is described. FIGS. 3A and 3B describe a method of the present invention wherein a stent, such as stent 10 of FIGS. 1 and 2, is compressed onto delivery wire 32 and preloaded into loader tube 50. FIGS. 3C to 3E describe use of the loader tube and delivery wire, preloaded with stent 10, in conjunction with sheath 40 of the present invention.

Referring to FIGS. 3A and 3B, stent 10 is shown wrapped around the winding portion of delivery wire 32. Proximal portion 12 preferably is wrapped around delivery wire 32 using guide 35 to control the pitch and wrap angle. Guide 35 defines helical ledge 39 that controls the pitch and overlap of adjacent turns of the vascular prosthesis during winding to the contracted delivery configuration. Either a proximal or distal edge of the vascular prosthesis may be abutted against helical ledge 39, with proximal stop 38 locating the proximal end of vascular prosthesis 10. When disposed within loader tube 50 (FIG. 3B), the length of the vascular prosthesis is the same as the length of the vascular prosthesis in the deployed configuration.

The specific steps for winding the vascular prosthesis onto delivery wire 32 in a proximal to distal direction are as follows: First, the tail of helical portion 12 of the stent is located and fixed at the proximal end of winding section 34 with the distal edge of the stent abutted against helical ledge 39. Next, helical portion 12 is wrapped around the delivery wire using the helical ledge to control the pitch and overlap of the turns. Loader tube 50 then is advanced over the vascular prosthesis to retain the helical portion in the contracted position on delivery wire 32. If the stent includes anchor portion 14, as depicted in FIGS. 1 and 2, the anchor portion of the stent is crimped down, and the loader tube is advanced over the anchor portion to retain the stent in the contracted delivery configuration. The loader tube and delivery wire, with pre-loaded stent, then may be packaged and sterilized for use.

Alternatively, stent 10 may be wound onto delivery wire 32 in a distal to proximal direction, as follows: First, the anchor portion of the stent is placed on delivery wire in a desired location, and the joint between anchor portion and the helical body portion of the stent is temporarily fixed to the inner member. Next, the helical portion of the stent is wrapped around the delivery wire in abutment to the helical ledge of the delivery wire. When the stent is completely wrapped around the delivery wire, loader tube 50 is advanced over the stent while rotating the loader tube in the direction in which the stent is wound. The loader tube then is advanced up to the joint where the anchor portion joins the helical portion. Next, the anchor portion is compressed into contact with the delivery wire and the loader tube is again advanced, while being rotated in the direction of the wrap, until it covers the anchor portion. The loader tube and delivery wire, with pre-loaded stent, then may be packaged and sterilized for use.

When the stent is loaded in accordance with the foregoing method, helical ledge 39 not only mitigates foreshortening, but in addition, prevents the proximal edge of the stent from sliding in the proximal direction during stent deployment.

When disposed in loader tube 50, vascular prosthesis 10 is constrained within lumen 51 so that it cannot expand or unwind during sliding translation of delivery wire 32 within the loader tube. Hemostatic valve 32 may be used to lock delivery wire 32 in position in loader tube 50 until it is desired to deploy the vascular prosthesis.

A method of stenting a target location within a vessel is now described. First, a conventional guide wire is advanced into a patient's vessel under fluoroscopic guidance until the distal tip is disposed at the target location, e.g., having a stenosis or aneurysm. Generally, if angioplasty of the stenosis is to be performed, a balloon catheter then is inserted along the guide wire and inflated to disrupt the stenosis. The balloon catheter then is deflated and the balloon catheter is withdrawn, leaving the guide wire in place.

Sheath 40 then is advanced over the guide wire so that atramautic tip is positioned at the target location. This may be determined, for example, by injecting radiographic dye through lumen 41 or by direct visualization of radiopaque marker 46. Once the distal end of the sheath is at the desired location, the conventional guide wire is withdrawn, leaving the sheath in place.

Next, as shown in FIG. 3C, loader tube 50 is coupled to the proximal end of sheath 40 using couplings 44 and 54. Hemostatic valves 45 and 53 are opened, and delivery wire 32 is urged in the distal direction, pushing stent 10 from lumen 51 into lumen 41. As described hereinabove, because the diameter of lumen 41 is substantially the same as the diameter of lumen 51, stent 10 remains compressed on delivery wire 32. Once stent 10 is transferred into lumen 41 of sheath 40.

Referring to FIGS. 3D and 3E, delivery wire 32 is advanced through lumen 41 of sheath 40 until stent 10 is aligned with lesion L in the target location, for example, as determined by fluoroscopic visualization of distal marker 37 and proximal stop 38 on delivery wire 32. Once proper alignment of the stent with the lesion is confirmed, delivery wire 32 is held stationary and sheath 40 is retracted proximally, as depicted in FIG. 3E, until stent 10 is deployed from within sheath 40.

More specifically, as sheath 40 is retracted proximally, anchor section 14 of the stent self-expands into engagement with the vessel wall within or distal to lesion L. When released from the constraint provided by the sheath, the struts of anchor section 14 expand in a radial direction to engage the interior of vessel V. After anchor section 14 is secured to the vessel wall distal of lesion L, sheath 40 is further retracted proximally to cause helical section 12 to unwind and deploy to its predetermined shape within vessel V. Once the last turn of the helical section is deployed, sheath 40 is withdrawn from the patient's vessel. Delivery wire 32 may be removed, or alternatively used as a guide wire for a balloon catheter to be inserted into the vessel to further expand the stent, if desired.

Referring now to FIG. 4, the problem of stent foreshortening as heretofore encountered with ribbon-type stents is described. As used in this specification, “foreshortening” refers to the length change of the stent between its contracted delivery configuration and its expanded deployed configuration. More specifically, the contracted delivery configuration, depicted in the upper portion of FIG. 4, corresponds to the state wherein consecutive turns of the stent have been tightly wrapped around adjacent turns to reduce the diameter of the stent to diameter d₁ and length of L₁, suitable for transluminal delivery to a target location within a vessel. In the deployed configuration, the stent is permitted to expand to its nominal working diameter, and has a diameter d₂ and length of L₂, suitable for supporting a target location within a vessel. “Foreshortening” is defined as the difference between the lengths L₁ and L₂.

In most interventional procedures, satisfactory stent placement requires predictable placement of the distal and proximal ends of the stent within a target vessel. Previously-known ribbon-type self-deploying stents, however, have encountered limited clinical acceptance due to problems associated with foreshortening and inaccurate placement.

Specifically, previously-known ribbon-type stents often are wound down around a delivery catheter in either an “edge to edge” manner (where the edges of adjacent turns lie next to one another) or with an overlap (or “shingled”), and then covered with a sheath that restrains the stent in the contracted delivery configuration. When wound “edge to edge,” the stent may be significantly longer in the contracted delivery configuration than in the deployed configuration, and thus result in significant foreshortening when deployed.

On the other hand, when the turns of the stent are permitted to overlap in the contracted delivery configuration, the turns of the stent may lock or bind within the delivery system during deployment. Further still, in either method of contracting the stent to its contracted delivery configuration, the stent has a tendency to jump or hop forwards or backwards when deployed, resulting in poor control. Thus, previously-known ribbon-type stents generally are perceived to be capable of less accurate deployment than conventional balloon expandable stents.

Guide 35 of delivery wire 32 of the present invention resolves this problem by controlling winding of the stent to a predetermined contracted delivery configuration, and likewise controlling unwinding of the stent during deployment to mitigate foreshortening.

In accordance with the principles of the present invention, it has been discovered that certain trigonometric relationships may be utilized whereby the sent may be wrapped to its reduced delivery diameter, and experience little or no foreshortening during deployment. These relationships are derived below, and then implemented in the delivery catheters of the present invention, as set forth below.

Referring now to the lower portion of FIG. 4, a previously-known ribbon-type stent is depicted in an unrolled, flattened configuration. When deployed, as schematically depicted by the single turn in the upper portion of the FIG. 5, stent 60 comprises a strip of material wrapped cylindrically at a diameter (d) over an axial length (L) for a number of revolutions (n). The strip is wrapped at a wrap angle (θ), which may be measured from a plane normal to the axis of the helix.

The strip has a width (ω) and an edge length (E); these are physical characteristics of the stent that do not change. On the other hand, the diameter (d), wrap angle (θ), number of revolutions (n), and axial length (L) are interrelated characteristics that vary depending upon the helical configuration of the stent. For example, the diameter of the stent varies between the contracted delivery configuration and deployed configuration, which also may effect the wrap angle, number of revolutions, and axial length.

From inspection of FIG. 5, it can be seen that the axial length of the stent in the helical configuration is L plus the proximal-most part of the projected strip width. This additional length may be computed as depicted in FIG. 6, using a right triangle in which one leg is the strip width (ω), and the hypotenuse is the strip width projected onto the helical axis of the stent. Because the angle on the right side of this triangle is equal to the wrap angle (θ), the strip width projected onto the helical axis of the stent is equal to ω/cosθ. The total length of the stent in the helical configuration is therefore L+ω/cosθ. In addition, it will be observed that, as wrap angle θ increases, the projected width of the strip also increases.

Referring now to FIGS. 4 and 5, foreshortening may be computed as the change in the axial length of the stent as it transitions from one diameter (d₁) to another (d₂) during deployment:
F=(L₁+ω/cos θ₁)−(L₂+ω/cos θ₂)
F=(L₁−L₂)+(ω/cos θ₁−ω/cos θ₂)
From inspection of FIG. 6, it can be seen that edge length E, axial length L and wrap angle θ are related by trigonometric relationship, and substituting these relationship into foregoing equation for foreshortening provides:
F=(E sin θ₁−E sin θ₂)+(ω/cos θ₁−ω/cos θ₂)
F=E(sin θ₁−sin θ₂)+ω(1/cos θ₁−1/cos θ₂)

Of primary interest in the context of the present invention is the case where there is no foreshortening (F=0) when the stent transitions from diameter d₁ to diameter d₂. By setting the above equation equal to zero, it will be observed that the edge component of the equation E(sin θ₁−sin θ₂) and the width component ω(1/cos θ₁−1/cos θ₂) must either be equal to zero, or be equal and opposite. For meaningful wrap angles (0<θ<90), both components will always have the same sign. Thus, in order for the equation to balance, both components of the equation must be equal to zero. This leads to the conclusion that for there to be no foreshortening during stent deployment, the two wrap angles must be equal: θ₁=θ₂. Accordingly, for a stent wrapped into a helical configuration, where strip width X and total edge length E are constant, the amount of foreshortening between two different configurations is dependant on wrap angle alone. Thus, to eliminate foreshortening between any two helical configurations, both configurations must have the same wrap angle θ.

Provision of the helical ledge directly on the exterior surface of the delivery wire as in the embodiment of FIG. 2 ensures zero foreshortening of the stent during deployment. Advantageously, the helical ledge also provides linear resistance to stent migration during advancement of delivery wire 32 and stent 10 through loader tube 50 and sheath 40, and also when sheath 40 is retracted during stent deployment. This engagement between the turns of the stent and the delivery wire maintains the linear stability of the stent, and reduces the risk that overlapping turns of the stent will bunch up or seize against the interior surface of the sheath. Moreover, the helical ledge ensures that the stent unwinds on its axis but does not experience significant linear change along the axis.

While preferred illustrative embodiments of the invention are described above, it will be apparent to one skilled in the art that various changes and modifications may be made therein without departing from the invention. The appended claims are intended to cover all such changes and modifications that fall within the true spirit and scope of the invention.

1. A delivery system for delivering a vascular prosthesis within a vessel, the vascular prosthesis having a contracted delivery configuration and a deployed configuration, the delivery system comprising:
a delivery wire;
a loader tube having a distal end and a lumen of a first diameter configured to constrain the vascular prosthesis to the delivery wire in the contracted delivery configuration; and
a sheath having a proximal end and a lumen of a second diameter, the second diameter dimensioned to accept the delivery wire and the vascular prosthesis while retaining the vascular prosthesis in the contracted delivery configuration; and
a coupling configured to engage the distal end of the loader tube to the proximal end of the sheath to enable the vascular prosthesis to be advanced from the loader tube to a target location within the vessel.

2. The delivery system of claim 1, wherein the coupling comprises male and female portions of a luer connector.

3. The delivery system of claim 1, wherein the lumen of the sheath further comprises a non-stick liner that defines the second diameter.

4. The delivery system of claim 1, wherein the proximal end of the sheath further comprises a hemostatic valve.

5. The delivery system of claim 1, wherein a proximal end of the loader tube further comprises a hemostatic valve.

6. The delivery system of claim 5, wherein the hemostatic valve is configured to selectively lock the delivery wire and the vascular prosthesis within the loader tube.

7. The delivery system of claim 1, wherein the delivery wire further comprises a winding section dimensioned to receive the vascular prosthesis.

8. The delivery system of claim 7, wherein the winding section comprises a guide that defines a pitch that facilitates consistent and accurate winding of the vascular prosthesis around the delivery wire.

9. The delivery system of claim 8, wherein the guide is configured to provide substantially zero foreshortening of the vascular prosthesis during deployment.

10. The delivery system of claim 1, further comprising at least one of a distal marker and a proximal stop disposed on the delivery wire.

11. The delivery system of claim 1, further comprising a proximal stop provided on the delivery wire to define a proximal boundary for the vascular prosthesis during mounting of the vascular prosthesis on the delivery wire.

12. The delivery system of claim 1 wherein the delivery wire further comprises an atraumatic tip.

13. The delivery system of claim 8 wherein the guide comprises a helical ledge.

14. The delivery system of claim 13 wherein the helical ledge comprises a helical coil affixed to the delivery wire.

15. The delivery system of claim 13 wherein the helical ledge comprises a larger diameter wire incorporated in a braided portion of the delivery wire.

16. The delivery system of claim 13 wherein the helical ledge comprises a reduced-diameter helical groove on the delivery wire.

17. A method for delivering a vascular prosthesis to a lesion within a patient's vessel, the vascular prosthesis having a contracted delivery configuration and a deployed configuration, the method comprising:
providing a delivery wire, a loader tube having a lumen and a sheath having a lumen;
compressing the vascular prosthesis onto the delivery wire to the contracted delivery configuration;
advancing the loader tube over the vascular prosthesis and the delivery wire to retain the vascular prosthesis in the contracted delivery configuration;
advancing the sheath within the patient's vessel to a target location;
coupling the loader tube to the sheath;
advancing the delivery wire and vascular prosthesis, in the contracted delivery configuration, from the loader tube and through the sheath to the target location; and
ejecting the vascular prosthesis from the sheath so that it deploys against the patient's vessel at the target location.

18. The method of claim 17, wherein ejecting the vascular prosthesis from the sheath comprises retracting the sheath proximally while holding the delivery wire stationary.

19. The method of claim 17, wherein the sheath includes a hemostatic valve, the method further comprising opening the hemostatic valve after coupling the loader tube to the sheath.

20. The method of claim 17, wherein the loader tube includes a hemostatic valve, the method further comprising opening the hemostatic valve prior to advancing the delivery wire and vascular prosthesis from the loader tube and through the sheath.

21. The method of claim 17, wherein the delivery wire includes a winding section, wherein compressing the vascular prosthesis onto the delivery wire comprises winding the vascular prosthesis onto the winding section.

22. The method of claim 21, winding the vascular prosthesis onto the winding section further comprises winding the vascular prosthesis onto the winding section with a pitch that reduces foreshortening of the vascular prosthesis during deployment.

23. The method of claim 17, wherein the sheath includes a radiopaque marker, the method further comprising advancing the sheath within the patient's vessel to the target location using the radiopaque marker to confirm a location of a distal end of the sheath.

24. The method of claim 17, wherein the delivery wire includes a radiopaque distal marker, the method further confirming a location of the delivery wire and vascular prosthesis at the target location using the radiopaque marker.

25. The method of claim 17, wherein advancing the sheath within the patient's vessel to the target location comprises advancing the sheath along a pre-positioned guide wire.

26. The method of claim 25 further comprising, prior to coupling the loader tube to the sheath, withdrawing the guide wire from the sheath while retaining the sheath stationary.
